(19) United States
(12) Patent Application Publication
(10) Pub. No.: US 2025/0009976 A1
(43) Pub. Date: Jan. 9, 2025

(54) STOPPER PLACEMENT IN A SYRINGE

(71) Applicant: AMGEN INC., Thousand Oaks, CA (US)

(72) Inventors: Fatemeh Fallahianbijan, Thousand Oaks, CA (US); Shermeen A. Abbas, Porter Ranch, CA (US); Albert Ju, Thousand Oaks, CA (US); Oscar Gonzales, Thousand Oaks, CA (US); Vikashni Padmakumar, Thousand Oaks, CA (US); Jeffrey A. Brown, Thousand Oaks, CA (US); Wael Mismar, Redondo Beach, CA (US)

(21) Appl. No.: 18/712,768

(22) PCT Filed: Dec. 2, 2022
(86) PCT No.: PCT/US2022/051598

§ 371 Date: May 23, 2024

Related U.S. Application Data
(60) Provisional application No. 63/285,789, filed on Dec. 3, 2021.

(51) Int. Cl. A61M 5/315

(52) U.S. Cl. CPC A61M 5/31515; A61M 2207/00

(57) ABSTRACT

A method of placing a plunger into a syringe barrel filled with a drug product. The method includes aligning a stopper with a longitudinal axis of a syringe barrel. The syringe barrel includes a proximal end, a distal end, and a reservoir. The drug product is disposed in the reservoir at the distal end. The method includes applying a vacuum pressure to the reservoir of the syringe barrel. The method includes pushing the stopper with a plunger rod into the reservoir of the syringe barrel to a first depth. The method includes removing the plunger rod from the reservoir of the syringe barrel after pushing the stopper to the first depth. The stopper migrates to a second depth that is different than the first depth in response to a pressure differential in the reservoir of the syringe barrel.

### CROSS-REFERENCE TO RELATED APPLICATION

Priority is claimed to U.S. Provisional Patent Application No. 63/285,789, filed Dec. 3, 2021, the entire contents of which are hereby incorporated by reference herein.

### FIELD OF DISCLOSURE

The present disclosure relates to a method of placing a stopper in a syringe, and more particularly, to a method of placing a stopper in a syringe with a low drug product fill volume.

### BACKGROUND

Syringes are medical delivery devices used to administer a drug product to a patient. Syringes are often marketed either in prefilled form, wherein a set dosage of drug product is already provided therein, or they are empty and intended to be filled from a vial or other source of medicament by an end user at the time administration of the medicament is desired. An example syringe 10 is shown in FIG. 1 and includes a barrel 11 adapted to retain a drug product in a reservoir 13. The barrel 11 includes a proximal end 11A, a distal end 11B, a flange portion 12, and the cavity or reservoir 13 extending between the proximal and distal ends 11A, 11B. The distal end 11B of the barrel 11 is often configured to include and/or mate with a conventional piercing element, such as a pointed needle cannula or a blunt ended cannula, to deliver the medicament contained in the barrel 11. For example, the syringe barrel distal end 11B may include a Luer lock component. The piercing element may be made of steel, plastic, or any other suitable material. A plunger rod may be inserted through the open proximal end 11a of the syringe barrel 11 and, through its engagement with an elastomeric or rubber-like stopper element fitted in a substantially fluid-tight manner within an interior wall 11C of the barrel 11, a user can apply manual force to the plunger to deliver the medicament through the piercing element. The syringe barrel proximal end 11A is open to receive the plunger rod and stopper component. The flange 12 is provided around the open distal end 11A of the syringe barrel 11 as a form of finger rest to facilitate a user's manipulation of the device.

It may be desirable, both for integrity of the medicament as well as for patient safety, to sufficiently sterilize the components of the syringe 10. External sterilization, for example, typically occurs after the prefilled syringe has been filled, fully assembled, and in at least some portion(s) of its final packaging. During sterilization, however, the stopper may migrate within reservoir 13 the barrel 11 from an initial position to a final position. In a typical prefilled syringe, a large portion of the reservoir 13 is filled with drug product, leaving little room for the stopper to migrate. However, in a syringe with a low drug product fill volume (i.e., there is a large portion of the reservoir 13 empty), the stopper is placed further down in the barrel 11, leaving more room for the stopper to migrate during the sterilization process. Thus, the initial placement of the stopper within the barrel 11 of the syringe 10 may account for migration during the sterilization process to ensure that the plunger migrates to an acceptable final position.

Known methods of stopper placement in prefilled syringes 10 include rod insertion or mechanical placement, vacuum assisted (“VA”) placement, and vacuum compression (“VC”) placement. However, each of these methods faces its own unique challenges when placing a stopper deep inside a syringe barrel with a low drug product fill volume. Mechanical stopper placement (e.g., plunger rod insertion) faces undesirable results because this method slows the rate at which stoppering can occur due to mechanical requirements of inserting and removing a component into each syringe barrel, thereby decreasing line throughput. Additionally, in some cases, mechanical insertion of a stopper can cause forced deformation of the stopper itself. For VA and VC methods, a vacuum applied to the syringe barrel 11 removes the headspace above the drug product to drive stopper placement into the reservoir 13. Particularly, the stopper is placed partially in the syringe barrel 11 at the flange 12, and a vacuum pressure within the barrel 11 drives movement of the stopper to a final depth. The VA method also includes compressing the stopper before inserting the stopper in the barrel 11, which results in drug product between the stopper ribs and plunger deformity. While the VC method may not lead to drug product in the stopper ribs, the VC process is much slower and is impractical when processing prefilled syringes in bulk. For both VA and VC methods, relying on the pressure differential to place the stopper deep in the reservoir 13 results in variable stopper placements and therefore poor process capability scores and longer migration times. Typically, the stopper reaches a final position after 12 to 24 hours, which delays the process of determining whether the prefilled syringes are suitable for use. Consequently, these methods lead to unacceptable rates of prefilled syringe rejections due to plunger deformity, drug product between stopper or plunger ribs, higher rates of stopper migration out of a target placement range-thereby increasing rates of prefilled syringe rejections. These rejections often occur at the sterilization location, different from the filling location, and therefore leads to inefficiency and waste.

### SUMMARY

The proposed methodology of stopper placement in a syringe with a low drug product fill volume involves more predictable and accurate results to place stoppers in prefilled syringes in acceptable ranges by supplementing known vacuum assist and vacuum compression techniques with a mechanical insertion component.

In accordance with a first aspect of the present disclosure, a method of placing a stopper into a syringe barrel partially filled with a drug product may include aligning a stopper with a longitudinal axis of a syringe barrel. The syringe barrel may include a proximal end, a distal end, and a reservoir. The drug product may be disposed in a distal end of the reservoir at the distal end of the syringe barrel. The method may include applying a vacuum pressure to the reservoir of the syringe barre. The vacuum pressure may be in a range of approximately 70 mBar to approximately 85 mBar. The method may further include pushing the stopper with a plunger rod into the reservoir of the syringe barrel to a first depth, thereby creating a pressure differential across the stopper. The first depth may be in a range of approximately 20 mm to approximately 40 mm from the distal end of the reservoir. Further, the method may include disengaging the plunger rod from the stopper after pushing the stopper to the first depth, thereby causing the pressure differential across the stopper to displace the stopper to a second depth different than the first depth.

In accordance with a second aspect of the present disclosure, a method of placing a stopper into a syringe barrel partially filled with a drug product may include aligning a stopper with a longitudinal axis of a syringe barrel. The syringe barrel may include a proximal end, a distal end, and a reservoir. The drug product may be disposed in a distal end of the reservoir at the distal end of the syringe barrel. The method may include applying a vacuum pressure to the reservoir of the syringe barrel and pushing the stopper with a plunger rod into the reservoir of the syringe barrel to a first depth, thereby creating a pressure differential across the stopper. The plunger rod may extend into the reservoir of the syringe barrel. Further, the method may include disengaging the plunger rod from the stopper after pushing the stopper to the first depth, thereby causing the pressure differential across the stopper to displace the stopper to a second depth different than the first depth.

In further accordance with any one or more of the foregoing first and second aspects, a method for placing a stopper in a syringe barrel may include any one or more of the following forms.

In one form, pushing the stopper may include inserting the plunger rod into the reservoir of the syringe barrel.

In another form, applying the vacuum pressure may at least partially coincide with pushing the stopper with the plunger rod into the reservoir of the syringe barrel.

In yet another form, pushing the stopper may include pushing the stopper into the syringe barrel a distance in a range of approximately 23 mm to 32 mm from the distal end.

In some forms, pushing the stopper may include pushing the stopper after applying the vacuum pressure to the syringe barrel.

In other forms, pushing the stopper may include pushing the stopper into the syringe barrel a distance in a range of approximately 30 mm to approximately 35 mm from the distal end of the reservoir.

In another form, aligning the stopper may include placing the stopper adjacent to the proximal end of the syringe barrel before inserting the stopper into the reservoir of the syringe barrel.

In one form, aligning the stopper may include compressing the stopper with a grip adjacent to the proximal end of the syringe barrel and pushing the compressed stopper with the plunger rod through the grip and into the reservoir of the barrel.

In some forms, applying the vacuum pressure may include evacuating a headspace above the drug product in the syringe barrel and applying a vacuum at the proximal end of the syringe barrel.

In other forms, the method may include removing the plunger rod from the reservoir of the syringe barrel.

In yet another form, applying the vacuum pressure to the syringe barrel may include applying a pressure in a range of approximately 70 mBar to approximately 85 mBar.

In one form, pushing the stopper may include pushing the stopper into the syringe barrel a distance in a range of approximately 20 mm to approximately 40 mm from a distal end of the reservoir.

### BRIEF DESCRIPTION OF THE DRAWINGS

FIG. 1 is a perspective view of a known syringe;

FIG. 2 is flowchart representative of a method of placing a stopper into the syringe of FIG. 1 having a low drug product fill volume in accordance with the teachings of the present disclosure;

FIG. 3 is a schematic diagram of a stopper insertion assembly and prefilled syringe having a low drug product fill volume in accordance with the teachings of the present disclosure, showing a stopper in an initial position;

FIG. 4 is the stopper insertion assembly and syringe of FIG. 3, showing the stopper in a partially inserted position;

FIG. 5 is the stopper insertion assembly and syringe of FIG. 3, showing the stopper at a first depth;

FIG. 6 is the stopper insertion assembly and syringe of FIG. 3, showing the stopper at the first depth with a plunger rod removed from the syringe;

FIG. 7 is the syringe of FIG. 3, showing the stopper at a final depth position;

FIG. 8 is a schematic diagram of a different stopper insertion assembly and prefilled syringe having a low drug product fill volume in accordance with the teachings of the present disclosure, showing a stopper in an initial position;

FIG. 9 is the stopper insertion assembly and syringe of FIG. 8, showing the stopper in a partially inserted position;

FIG. 10 is the stopper insertion assembly and syringe of FIG. 8, showing the stopper at a first depth;

FIG. 11 is the stopper insertion assembly and syringe of FIG. 8, showing the stopper at the first depth with a plunger rod removed from the syringe; and

FIG. 12 is the syringe of FIG. 8, showing the stopper at a final depth position.

### DETAILED DESCRIPTION

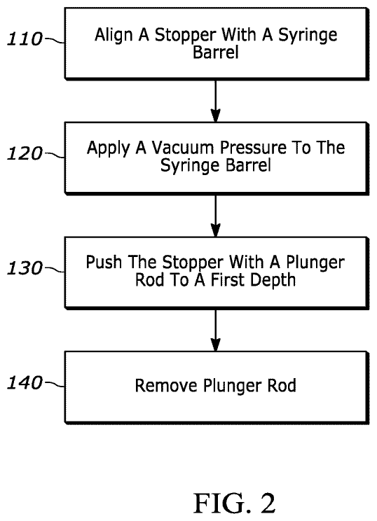

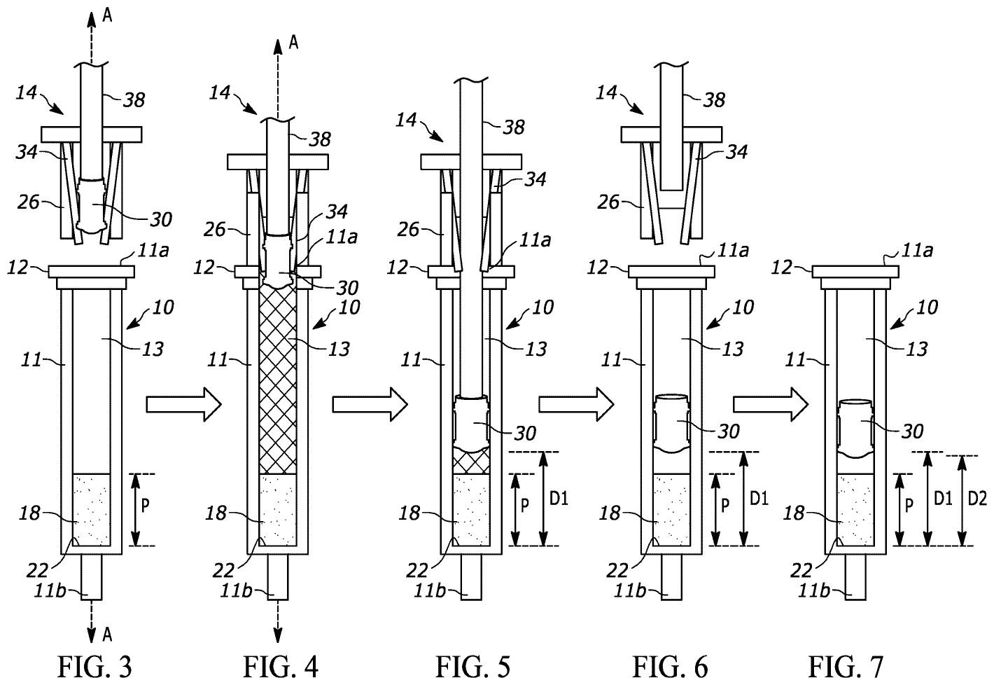

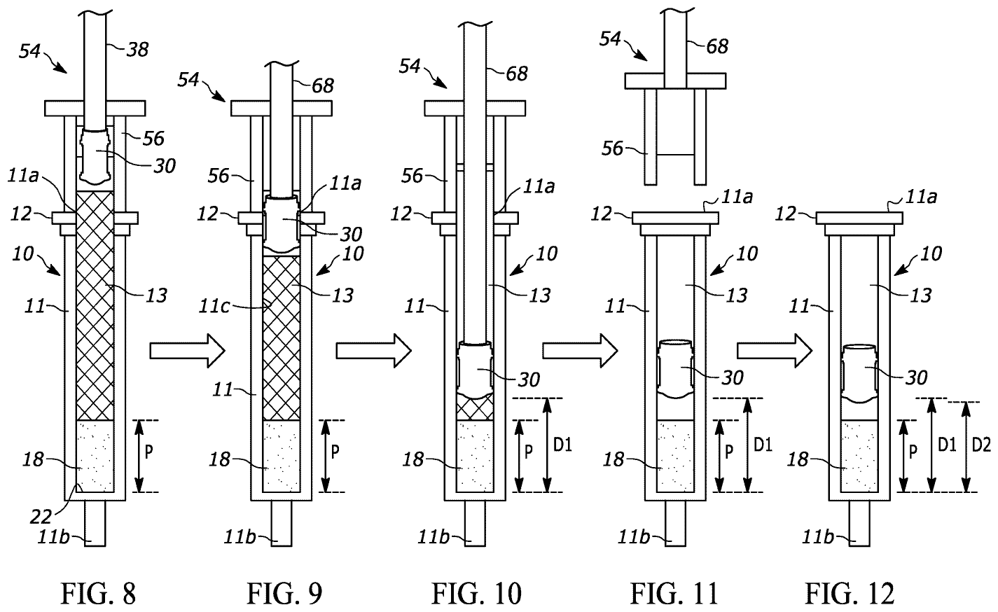

An example method 100 of placing a stopper in a syringe barrel having a low drug product fill volume of the present disclosure is depicted in the flow chart of FIG. 2. The example method 100 may result in higher poor process capability (“PPK”) values, lower rejection rates, and faster throughput compared to conventional stoppering methods. The method of FIG. 2 may be implemented through a single-stage process that includes Vacuum Assisted (“VA”) and mechanical insertion techniques, depicted in FIGS. 3-7, and a double-stage process that includes Vacuum Compression (“VC”) and mechanical insertion techniques, depicted in FIGS. 8-12. Both of the single-stage and double-stage processes utilize a vacuum and mechanical methods to drive the stopper to a target depth in a prefilled syringe having a low drug product fill volume.

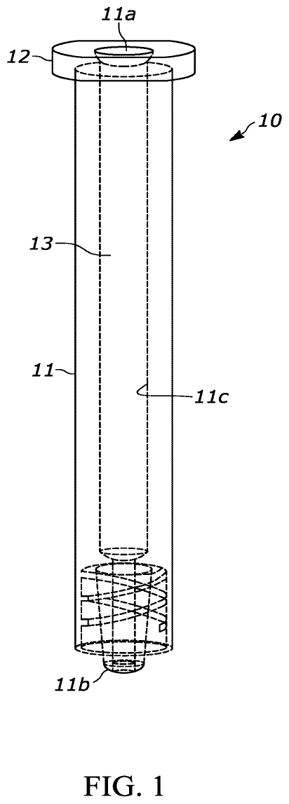

Generally speaking, both single-stage and double-stage processes may be carried out following the steps in the method 100 of FIG. 2. The method 100 integrates a vacuum pressure and mechanical rod insertion techniques to place a stopper deep inside a reservoir 13 of a syringe 10, such as the syringe of FIG. 1. A first step 110 of the method 100 includes aligning a stopper with a longitudinal axis A of the syringe barrel 11, and a second step 120 of the method includes applying a vacuum pressure to the reservoir 13 of the syringe barrel 11. Subsequently or simultaneously with applying the vacuum pressure to the syringe barrel 11, the method 100 includes a step 130 of pushing the stopper through the opening in the proximal end 11A of the syringe barrel 11 with a plunger rod. The plunger rod extends through the opening 11A and into the reservoir 13 to place the stopper at a first depth, which is in a range of approximately 20 mm to approximately 40 mm from a proximal end (i.e., near the flange 12) of the reservoir 13. Finally, the method 100 includes a step 140 of removing the plunger rod from the syringe barrel 11, thereby permitting the stopper to migrate to a second, final depth, different than the first depth, in response to a pressure differential in the reservoir 13 of the syringe barrel 11. In some embodiments, the plunger rod can be a component of an assembly device (e.g., an insertion assembly system) that is separate from the syringe, and which is used to place the stopper in the syringe barrel as explained. In such embodiments, the plunger rod is not part of an end product including the syringe, but rather, it is completely removed from the syringe after placing the stopper such that the syringe and stopper can be separated from the plunger rod for further processing and/or use. In other embodiments, the plunger rod can constitute one component of an end product that also includes the syringe such as a pre-filled syringe or other injector, for example.

In FIGS. 3-8, a single-stage process of placing a stopper into a syringe barrel at a target depth includes operating a stopper insertion assembly 14 with a prefilled syringe 10 having a low drug product fill volume 18. Turning first to FIG. 3, the initial step 110 of method 100 of aligning the stopper 30 with the syringe barrel 11 of the prefilled syringe 10 is illustrated. The prefilled syringe 10 is partially filled with a drug product 18 disposed at a distal end 22 of the reservoir 13 with a meniscus at a depth P. The stopper insertion assembly 14 includes a grip 26 or vice that holds a stopper 30 above the open, proximal end 11A of the syringe barrel 11 and aligns the stopper 30 with a longitudinal axis A of the syringe 10. The grip 26 may be part of a machine or automated system, such as a filling machine. The grip 26 includes an angled chute 34 that compresses the stopper 30 in the initial position to facilitate insertion of the stopper 30 into the syringe barrel 11.

In the single-stage process depicted in FIGS. 4 and 5, the step 120 of applying a vacuum pressure and the step 130 of pushing the stopper 30 into the syringe barrel 11 are performed together or partially together. The grip 26 engages the flange 12 of the syringe 10 and pulls a vacuum the headspace above the drug product 18 from the reservoir 13 as a plunger rod 38 of the assembly 14 pushes the stopper 30 in a distal direction through the chute 34, the proximal end 11A, and into the reservoir 13 of the barrel 11. The reservoir 13 of the syringe barrel 11 experiences a vacuum pressure in a range of approximately 70 mBar to approximately 85 mBar, and assists with the distal insertion of the plunger rod 38. In FIG. 5, the plunger rod 38 extends into the reservoir 13 of the syringe barrel 11, placing the stopper 30 at a first depth D1 above the meniscus P of the drug product 18 and in a range of approximately 23 mm to approximately 32 mm from the distal end 22 of the reservoir 13. In the illustrated example, the stopper insertion assembly 14 performs both steps of applying a vacuum and pushing the stopper 30 into the syringe barrel 11. Specifically, a vacuum is drawn through an associated stoppering bar attached to the filling machine. A vacuum line is attached to the bar and suction is achieved by removing the air in the bar and associated syringe barrel 11 while a vacuum bellows are in contact with the syringe barrel 11. If vacuum is not actively being drawn through the bar and attached syringe barrel 11 at the time of stoppering (e.g., step 130), compressed air within the syringe barrel 11 will push against the stopper placement and can result in the stopper popping out of place. To avoid this, providing a vacuum within the syringe barrel 11 equilibrates the pressure within the syringe barrel 11 over a short time period. However, in other examples, the steps of drawing a vacuum and pushing the stopper 30 may be performed by different assemblies or mechanisms sequentially or coincidentally. In FIG. 6, the plunger rod 38 and grip 26 of the stopper insertion assembly 14 disengage from the syringe barrel 11, and the plunger rod 38 moves in a proximal direction and out of the syringe barrel 11, leaving the stopper 30 at the depth D1. The remaining vacuum pressure between the drug product 18 and the stopper 30 drives the stopper 30 to a final depth D2 as shown in FIG. 7. The final depth D2 is in a range of approximately 30 mm to 35 mm. The stopper 30 relaxes to its initial size once in the syringe barrel 11 and at the final depth D2. In particular, the method 100 results in placing the stopper 30 at a final depth D2 of 34.1±0.27 mm from the distal end 22 of the reservoir for a 0.5 mL Terumo syringe when the plunger rod depth parameter is approximately 25.9 mm from the distal end 22 of the reservoir 13 and the vacuum pressure parameter is approximately 80 mBar.

The single-stage method of placing a stopper in a prefilled syringe having a low drug product fill volume as depicted in FIGS. 3-7 provides many advantages over current stoppering methods, and in particularly, the VA method. For example, the single-stage process decreases variation in final stopper position, thereby providing more uniform and predictable results, leading to less rejections of prefilled syringes after sterilization. A combination of the mechanical assertion and the vacuum assisted technologies provide a lower pressure differential within the reservoir compared to the VA method, for example. As a result, the stopper 30 migrates only slightly in comparison to reach the final depth, leading to minimal depth variability and more predictable placement in the target range. Additionally, with the plunger rod insertion assembly 24, the plunger rod 38 drives the stopper 30 closer to the final depth D2, and the time of migration of the stopper 30 from the initial depth D1 to the final depth D2 decreases significantly compared to the VA method. Moreover, the single-stage method described herein results in a lower potential risk of liquid in stopper ribs, as shown in Table 1 below.

Turning now to FIGS. 9-12, a double-stage process of placing a stopper into a syringe barrel at a target depth includes operating a stopper insertion assembly 54 with a prefilled syringe 10 having a low drug product fill volume 18. FIG. 8 illustrates the initial step 110 of method 100 of aligning the stopper 30 with the syringe barrel 11. The prefilled syringe 10 is partially filled with the drug product 18 disposed in the reservoir 13 with a meniscus at the depth P. In one example using a 0.5 mL syringe, the depth P is in a range of approximately 0.15 mL to approximately 0.2 mL. The stopper insertion assembly 54 includes a grip 56 or vice that holds the stopper 30 above the open, proximal end 11A of the syringe barrel 11 and aligns the stopper 30 with the longitudinal axis A of the syringe 10. The grip 56 may be part of a machine or automated system, such as a filling machine.

In the double-stage process, the method 100 includes the step 120 of applying a vacuum pressure to the reservoir 11 before the step 130 of pushing the stopper 30 into the syringe barrel 11. In FIG. 8, the grip 56 of the stopper insertion assembly 54 engages the flange 12 of the syringe 10 and pulls a vacuum from the reservoir 13, thereby removing a headspace from the syringe barrel 11. Applying a vacuum pressure in a range of approximately 70 mBar to approximately 85 mBar assists with the distal insertion of the plunger rod 68 through the syringe barrel 11, as shown in FIG. 9. In other words, the syringe barrel 11 is pressurized when performing the step 130 of pushing the stopper 30 in a distal direction with a plunger rod 68 of the insertion assembly 54. The plunger rod 68 is in sealing engagement with interior walls of the syringe barrel 11, and drives the stopper 30 through the grip 56 and open proximal end 11A of the syringe barrel 11. In FIG. 10, the plunger rod 58 extends into the reservoir 13 of the syringe barrel 11, pushing the stopper 30 to a first depth D1 above the meniscus of the drug product 18, and in a range of approximately 30 mm to approximately 35 mm from the distal end 22 of the reservoir 13. In the illustrated example, the stopper insertion assembly 54 performs the steps of drawing the vacuum and pushing the stopper 30 with a plunger rod 68. However, in other examples, the steps of drawing a vacuum and pushing the stopper 30 may be performed by different assemblies or mechanisms.

In FIG. 11, the plunger rod 68 and grip 56 of the stopper insertion assembly 54 disengage from the syringe barrel 11, and the plunger rod 68 moves in a proximal direction and out of the syringe barrel 11. The remaining vacuum pressure between the drug product 18 and the stopper 30 drives the stopper 30 to a final depth D2 as shown in FIG. 12. The final depth D2 is in a range of approximately 30 mm to 35 mm. In particular, the method 100 results in placing the stopper 30 at a final depth D2 of 34.6±0.14 mm from the distal end 22 of the reservoir 13 when the plunger rod depth parameter is approximately 34.4 mm from the distal end 22 of the reservoir 13 and the vacuum pressure parameter is approximately 80 mBar.

The double-stage method of placing a stopper in a prefilled syringe having a low drug product fill volume as depicted in FIGS. 9-12 provides many advantages over current stoppering methods, and in particularly, the VC method. For example, the double-stage process decreases variation in final stopper position, thereby providing more uniform and predictable results, leading to less rejections of prefilled syringes after sterilization. A combination of the mechanical assertion and the vacuum compression technologies provide a lower pressure differential within the reservoir compared to a VC method, for example. As a result, the stopper 30 migrates only slightly in comparison to reach the final depth D2. Additionally, with the plunger rod insertion assembly 54, the plunger rod 68 drives the stopper 30 closer to the final depth D2, and the time of migration of the stopper to the final depth decreases significantly. Moreover, the double-stage method described herein results in a lower risk of plunger deformity, as shown in Table 1 below.

Table 1 below compares known methods (e.g., VA and VC) with the methods described herein (e.g., single-stage or VA+rod insertion method and double-stage or VC+rod insertion method) resulting in improved performance for the methods described herein. Specifically, the single-stage and double-stage methods depicted in FIGS. 3-8 and FIGS. 9-12, respectively, obtain greater PPK values and line speed, and lower rates of plunger migration, deformation, and final depth variability.

TABLE 1
Stopper Placement Technology Comparison

| | | | | | | Plunger Depth |
|---|---|---|---|---|---|---|
| Plunger | Final | | | | | after 20 minutes |
| Placement | Plunger | | Throughput | Line Speed | Defect/Plunger | in mm |
| Technology | Depth (mm) | PPK | (main-drive) | Syringes/hour | Deformation | (Target: 34.4 mm) |
| VA (75 mBar) | 34.8 ± 0.35 | 3.17 | 100% | 2337 | Liquid in ribs | 34.72 |
| | | | | | (1.2%) | |
| VC (80 mBar) | 34.4 ± 0.12 | 8.3 | 40% | NA | Plunger deformity | 34.16 |
| | | | | | (4.2%) | |
| VA + Rod Insertion | 34.1 ± 0.27 | 3.7 | 100% | 1690 | Liquid in ribs | 33.58 |
| (80 mBar & 25.9 mm) | | | | | (0.6%) | |
| VC + Rod Insertion | 34.6 ± 0.14 | 7.7 | 40% | 863 | Plunger deformity | 34.54 |
| (80 mBar & 34.4 mm) | | | | | (<0.1%) | |

As shown in Table 1, above, the single-stage and double-stage methods disclosed herein result in improved standard deviation of final depth. For example, with VA methods, the final plunger depth is approximately 34.8±0.35 mm compared to the single-stage method disclosed herein of approximately 34.6±0.14 mm. This standard deviation is smaller for VC+rod insertion method, but the defect rate is higher for VC only.

There are several factors governing the performance of plunger placement process: plunger position variation, plunger deformity/defect rate, and filling and stoppering line speed (i.e., number of syringes filled and plungered per unit of time). The PPK values of the single-stage method disclosed herein improved compared to VA method, as shown in Table 1 above. For example, with VA methods, the PPK value is 3.17, whereas the single-stage method disclosed herein resulted in a PPK value of 3.7. Although the VC only method shows larger PPK, a better performance may be achieved with VC+rod insertion. For each method listed in Table 1 (except VC only), 800 syringes were filled and stoppered. The defect rate was so high that a large sample size was not generated for the VC only method.

Additionally, line speed for methods VA+rod insertion was reduced as a result of the time needed to physically insert the stoppering rods into the syringes. For example, manually placing bottle caps on top of bottles may be much quicker than manually pushing each bottle cap into the neck of the bottle. By speeding up the process to match the rate of the non-insertion rod approach, there may be a high probability of introducing defects or machine errors due to the rapid operating pace and quick movements. Under ideal conditions, a faster operating line speed may be beneficial to reach an increased throughput. While plunger depth migration is faster, the line speed may be slower to avoid defects and machine errors.

Significantly, the disclosed single- and double-stage methods disclosed herein result in much faster stopper migration times. Typically, the stopper reaches a final depth after 12 to 24 hours using VA or VC methods. By comparison, the stopper reaches a final depth in 5 to 20 minutes using the single-stage and double-stage methods disclosed herein. Plunger depth measurements were taken 20 minutes after stoppering, and the results show that the plunger reached 99% of the target depth.

Rejections due to stopper deformity and drug product between stopper ribs also decreased significantly. For example, in VA only method, 1.2% of the prefilled syringes have liquid in the stopper ribs, and in VC only method, 4.2% of the prefilled syringes result in plunger deformity. Rejections due to liquid in the stopper ribs and plunger deformity decreased, comparatively, when a mechanical insertion component was integrated with each vacuum process. For example, when mechanical insertion is paired with VA, instances of liquid in the stopper ribs decreased to 0.6% of prefilled syringes. Additionally, when mechanical insertion is paired with VC, plunger deformity decreased to less than 0.1% of prefilled syringes. The occurrence rate of the deformation decreases, and the severity of a “bend” in a primary rib of the stopper was also less pronounced.

Below in Table 2, the single-stage and double-stage methods described herein are compared. As shown in the table, other factors may be considered when determining the appropriate “recipe” for plunger placement when using these methods. For example, the insertion rod speed, aeration duration, and vacuum start stopper time and pressure may be modified to achieve more accurate results. Insertion rod speed refers to the rate at which the rod physically moves. Insertion position versus final plunger depth may vary due to equilibration of vacuum force within the syringe barrel. As an example, if a stopper was physically placed just inside the top of a syringe barrel but had a strong vacuum force applied to the interior of the syringe, the stopper would tend to travel deeper into the syringe beyond the initial placement stage as the pressure equilibrates.

TABLE 2
Vacuum and Mechanical Processes for Inserting a Stopper Comparison

| | | Insertion | | | Aeration | | |
|---|---|---|---|---|---|---|---|
| | Vacuum | Position | | Insertion rod | Duration | Vacuum start | |
| Method | (mBar) | (mm) | | speed | (ms) | stopper | |
| VA + Rod Insertion | 80 | −150 | mm | 5% | 40 | 0 | ms |
| | | −154 | mm | | | 100 | mBar |
| VC + Rod Insertion | 80 | −144.5 | mm | 5% | 60 | 350 | ms |
| | | −142 | mm | | | 100 | mBar |

The stopper placement method 100 of FIG. 2, which may be either a single-stage or double-stage process, places a stopper deep inside a syringe barrel 11 containing a drug product using vacuum and mechanical placement methods. For example, the syringe 10 may be a 0.5 mL Terumo syringe. However, the method 100 described herein may be useful for a variety of syringe sizes (e.g., 1 mL syringes, 2.25 mL syringes, 5 mL cartridges, etc.) filled with a drug product that occupies less than half of the volume of the syringe barrel reservoir.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-?467 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNF? monoclonal antibody); Reopro® (abciximab, anti-GP Ilb/llia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-? 4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein); VEGF trap (Ig domains of VEGFR1 fused to IgG1 Fc); Zenapax® (daclizumab); Zenapax® (daclizumab, anti-IL-2R? mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNF? mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-? 5?1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFN? mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCG? mAb (MDX-1307); anti-mesothelin dsFv-PE38 conjugate (CAT-5001); anti-PD1mAb (MDX-1106 (ONO-4538); anti-PDGFR? antibody (IMC-3G3); anti-TGFß mAb (GC-1008); anti-TRAIL Receptor-2 human mAb (HGS-ETR2); anti-TWEAK mAb; anti-VEGFR/FIt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) molecules such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF? monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)-N-((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla®(apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein (a), also known as Lp (a), or another product containing a small interfering RNA (siRNA) that lowers lipoprotein (a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA) x anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF 15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BITE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vill (EGFRvIII) BiTE® (bispecific T cell engager) molecule. In some embodiments, the drug delivery device may contain or be used with AMG 673 or another product containing a half-life extended (HLE) anti-CD33×anti-CD3 BITE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A method of placing a stopper into a syringe barrel partially filled with a drug product, the method comprising:
aligning a stopper with a longitudinal axis of a syringe barrel, the syringe barrel including a proximal end, a distal end, and a reservoir, wherein the drug product is disposed in a distal end of the reservoir at the distal end of the syringe barrel;
applying a vacuum pressure to the reservoir of the syringe barrel, the vacuum pressure in a range of approximately 70 mBar to approximately 85 mBar;
pushing the stopper with a plunger rod into the reservoir of the syringe barrel to a first depth, thereby creating a pressure differential across the stopper, the first depth being in a range of approximately 20 mm to approximately 40 mm from the distal end of the reservoir; and
disengaging the plunger rod from the stopper after pushing the stopper to the first depth, thereby causing the pressure differential across the stopper to displace the stopper to a second depth different than the first depth.

2. The method of claim 1, wherein pushing the stopper includes inserting the plunger rod into the reservoir of the syringe barrel.

3. The method of claim 1, wherein applying the vacuum pressure at least partially coincides with pushing the stopper with the plunger rod into the reservoir of the syringe barrel.

4. The method of claim 3, wherein pushing the stopper includes pushing the stopper into the syringe barrel a distance in a range of approximately 23 mm to 32 mm from the distal end.

5. The method of claim 1, wherein pushing the stopper includes pushing the stopper after applying the vacuum pressure to the syringe barrel.

6. The method of claim 5, wherein pushing the stopper includes pushing the stopper into the syringe barrel a distance in a range of approximately 30 mm to approximately 35 mm from the distal end of the reservoir.

7. The method of claim 1, wherein aligning the stopper includes placing the stopper adjacent to the proximal end of the syringe barrel before inserting the stopper into the reservoir of the syringe barrel.

8. The method of claim 1, wherein aligning the stopper includes compressing the stopper with a grip adjacent to the proximal end of the syringe barrel and pushing the compressed stopper with the plunger rod through the grip and into the reservoir of the barrel.

9. The method of claim 1, wherein applying the vacuum pressure includes evacuating a headspace above the drug product in the syringe barrel and applying a vacuum at the proximal end of the syringe barrel.

10. The method of claim 1, further comprising removing the plunger rod from the reservoir of the syringe barrel.

11. A method of placing a stopper into a syringe barrel partially filled with a drug product, the method comprising:
aligning a stopper with a longitudinal axis of a syringe barrel, the syringe barrel including a proximal end, a distal end, and a reservoir, wherein the drug product is disposed in a distal end of the reservoir at the distal end of the syringe barrel;
applying a vacuum pressure to the reservoir of the syringe barrel;
pushing the stopper with a plunger rod into the reservoir of the syringe barrel to a first depth, thereby creating a pressure differential across the stopper, the plunger rod extending into the reservoir of the syringe barrel;
disengaging the plunger rod from the stopper after pushing the stopper to the first depth, thereby causing the pressure differential across the stopper to displace the stopper to a second depth different than the first depth.

12. The method of claim 11, wherein applying the vacuum pressure to the syringe barrel includes applying a pressure in a range of approximately 70 mBar to approximately 85 mBar.

13. The method of claim 11, wherein pushing the stopper includes pushing the stopper into the syringe barrel a distance in a range of approximately 20 mm to approximately 40 mm from a distal end of the reservoir.

14. The method of claim 11, wherein applying the vacuum pressure at least partially coincides with pushing the stopper with the plunger rod into the reservoir of the syringe barrel.

15. The method of claim 11, wherein pushing the stopper includes pushing the stopper after applying the vacuum pressure to the syringe barrel.

16. The method of claim 14, wherein pushing the stopper includes pushing the stopper into the syringe barrel a distance in a range of approximately 23 mm to 32 mm from a distal end of the reservoir.

17. The method of claim 15, wherein pushing the stopper includes pushing the stopper into the syringe barrel a distance in a range of approximately 30 mm to approximately 35 mm from a distal end of the reservoir.

18. The method of claim 11, wherein aligning the stopper includes (a) placing the stopper adjacent to the proximal end of the syringe barrel before inserting the stopper into the reservoir of the syringe barrel, and/or (b) compressing the stopper with a grip adjacent to the proximal end of the syringe barrel and pushing the compressed stopper with the plunger rod through the grip and into the reservoir of the barrel.

19. (canceled)

20. The method of claim 11, wherein applying the vacuum pressure includes evacuating a headspace above the drug product in the syringe barrel and applying a vacuum at the proximal end of the syringe barrel.

21. The method of claim 11, further comprising removing the plunger rod from the reservoir of the syringe barrel.
